(19) United States
(12) Patent Application Publication
(10) Pub. No.: US 2005/0106111 A1
(43) Pub. Date: May 19, 2005

(54) Compositions and methods for reducing dental plaque

(75) Inventor: Trevor P. Castor, Arlington, MA (US)

Correspondence Address: PERKINS, SMITH & COHEN LLP, ONE BEACON STREET, 30TH FLOOR, BOSTON, MA, 02108, US

(73) Assignee: Aphios Corporation

(21) Appl. No.: 10/942,485

(22) Filed: Sep. 16, 2004

Related U.S. Application Data
(60) Provisional application No. 60/503,383, filed on Sep. 16, 2003.

(51) Int. Cl. A61K 7/16

(52) U.S. Cl. 424/49

(57) ABSTRACT

Described herein are compositions and methods for preventing and reducing the formation of dental plaque. Also described herein are methods of producing the compositions used to prevent or reduce dental plaque.

### RELATED APPLICATIONS

This application claims priority to and the benefit of U.S. Provisional 60/503,383, filed Sep. 16, 2003.

### FIELD OF THE INVENTION

The present invention relates generally to dental health care. In particular, the instant invention pertains to compositions and methods for reducing dental plaque.

### BACKGROUND OF THE INVENTION

The mammalian tooth surface is colonized by a large number of microorganisms that constitute the normal flora of the mouth. The colonization of the tooth surface occurs in a sequence of events that occur immediately after a professional teeth cleaning. First, a proteinaceous pellicle develops on the tooth surface, consisting primarily of host-derived proline-rich proteins, salivary α-amylase, mucin, and statherin. These proteins provide attachment sites for the first layer of bacterial colonizers. The primary colonizers are streptococci, like Streptococcus oralis, Streptococcus gordonii, and Streptococcus sanguis, and actinomyces, like Actinomyces naeslundii. Streptococci and actinomyces carry receptors on their cell surfaces that allow them to attach to the pellicle on the tooth surface as well as to each other. With subsequent growth of the cells, a dense microbial layer (referred to as plaque) forms distal to the pellicula layer.

The plaque layer continues to grow in thickness by accretion of other bacterial species that possess cell surface receptors for streptococci and actinomyces. This second layer consists of other early colonizers that include Propionibacterium acnes, Prevotella loeschii, and Veillonella atypica. Particularly important in this layer is Fusobacterium nucleatum, a large rod that is capable of initiating contacts with early colonizers like A. naeslundii and V. atypica, as well as with late colonizers. These interactions between cocci and cocci, and cocci and rods have been observed in electron micrographs of dental plaque and can be detected in tube-based coaggregation assays.

The rate of plaque formation can be reduced by the regular use of dentifrices, but this in itself is not sufficient. In order to maintain healthy teeth, a professional, usually a dental hygienist, must mechanically remove the plaque layer. When the teeth are not adequately cared for, gingivitis can result. Gingivitis is produced due to further bacterial layers that have formed on the basal layers. Typically, the late colonizers associated with gingivitis are Porphyromonas gingivalis and Selenomonas flueggei. In teeth that have not been adequately cared for, gingivitis and root caries can lead to systemic infections resulting from the transport of bacteria into the circulatory system. Possible complications include endocarditis. The control of plaque development is therefore important for general health and well-being of the individual, but is not given enough emphasis by drug researchers/developers and pharmaceutical companies.

Gingivitis is a concern for a large number of people. Epidemiological surveys indicate that an average of 50% of the adult population of the United States (US) has gingivitis. Gingivitis is characterized by gingival inflammation and/or bleeding and is caused by plaque at and under the gingival margins. Most people brush their teeth; however toothbrushes cannot effectively remove plaque at or under the gum line. Floss is effective at removing plaque in difficult to reach locations; however only about 20% of the US population uses floss. Inconvenience is a commonly cited reason for not flossing. Since gingivitis is caused by plaque and plaque is composed of various kinds of bacteria, in theory anti-microbial agents should be effective against gingivitis.

There are a number of anti-microbial agents formulated in toothpaste or rinses on the market. The most effective of these agents is chlorhexidine digluconate (CHG). CHG reduced gingivitis by 50-80% in clinical trials. However, CHG is available in the US by prescription only and is generally used on a short-term basis (2-4 weeks only). Patient compliance is generally poor due to the unpleasant side effects associated with the use of CHG, which include staining of the teeth, interference with taste function, and enhanced calculus formation. Two products available on the over-the-counter (OTC) market have shown marginal effectiveness in clinical trials, Total® and Listerine®. Total, a toothpaste containing triclosan, reduced gingivitis by 20-25% in clinical trials. Listerine reduced gingivitis by 20-35% in clinical trials. Neither triclosan nor Listerine are substantive agents, thus the anti-microbial effect is lost quickly. The remaining anti-microbial agents available in OTC products have failed to show effectiveness in clinical trials. Thus, there is currently no truly efficacious anti-gingivitis product that is also both convenient to use and appealing to the consumer. It appears that the only chemicals that have been shown to have potent anti-plaque and anti-cariogenic activity are fluoride and chlorhexidine, both of which are halogenated. Other commonly used compounds including the phenolics are not as effective as plaque- and caries-control agents.

Considering the importance of healthy teeth and gums in general human health, a broader systematic search for plaque- and caries-control compounds is justified, and tapping into a new biological resource would be worthwhile.

### SUMMARY

The present invention relates to compositions and methods for preventing and reducing the formation of dental plaque. This invention also pertains to methods of producing the compositions used to prevent or reduce dental plaque.

One embodiment of the present invention pertains to compositions that reduce and/or prevent the formation of dental plaque. In one aspect, the compositions are anti-microbial agents derived from marine organisms. These anti-microbial agents can be selected from the group consisting of prodigiosin, magnesidin and homologs thereof.

One embodiment of the present invention pertains to methods for preventing or reducing the formation of dental carries and/or plaque. The methods disclosed herein comprise contacting the oral cavity, including teeth housed within the cavity, with one or more compositions of the present invention.

Another embodiment of the present invention pertains to methods for producing the marine anti-microbial compositions using supercritical fluid technology. This extraction technology facilitates the purification of anti-microbial agents from marine microbe sources.

### BRIEF DESCRIPTION OF THE DRAWINGS

FIG. 1 is an illustration of a supercritical fluid extraction apparatus;

FIG. 2 is a graph illustrating supercritical fluid phase;

FIG. 3 is a mass spectral scan;

FIG. 4 is a proton NMR spectrum;

FIG. 5 is a COSY NMR spectrum;

FIG. 6 is an HPLC chromatogram of C6 homolog; and

FIG. 7 is an HPLC chromatogram of C4 homolog.

### DETAILED DESCRIPTION

The present invention relates to compositions and methods for preventing and reducing the formation of dental plaque. This invention also pertains to methods of producing the compositions used to prevent or reduce dental plaque.

It appears that only chemicals that demonstrate potent anti-plaque and anti-cariogenic activity are fluoride and chlorhexidine, both of which are halogenated. Marine microorganisms are a unique source of novel genotypes and chemical entities, which can generate compounds that integrate halogens such as chlorine and bromine. Most commercial anti-plaque compounds, e.g., triclosan, chlorohexidene and fluorine, are halogenated. Marine microorganisms, because of their intrinsic capability to metabolize halogens, can thus be an important resource for the discovery of bioactive secondary metabolites and compounds with anti-microbial, anti-cariogenic and anti-plaque forming properties.

Marine microorganisms are being increasingly looked to as a source of novel bioactive compounds, and several reviews on the subject have appeared recently (Fenical, 1993; Okami, 1993; Fenical and Jensen, 1993; Jensen and Fenical, 1994; and Davidson, 1995). Much of the emphasis, however, is on anti-tumor and antibiotic activity. For example, the anti-tumor drugs octalactin (Tapiolas et al., 1991) and altemicidin (Takahashi et al., 1989) have been isolated from marine Streptomyces spp. Urauchamycin is an antimicrobial produced by Streptomyces, while andrimid and moiramide are anti-microbials isolated from Pseudomonas fluorescens (Needham et al., 1994). Anti-fungals have been described in the archaebacterium Thermococcus (Ritzau et al., 1993), and in symbiotic bacteria associated with lobster embryos (Gil-Turnes et al., 1989). Other bioactives isolated from marine microbes include a bronchodilator produced by Alteromonas rubra (Holland et al., 1984). Only two studies describe potential anti-plaque compounds from a marine source—seaweed (Kubo et al., 1992; and Saeki, 1994).

One embodiment of the present invention pertains to compositions that reduce and/or prevent the formation of dental caries. In one aspect, the compositions are anti-microbial agents derived from marine organisms. In one aspect, the marine organism is Vibrio gazogenes. These marine-derived anti-microbial agents can be selected from the group consisting of prodigiosin, magnesidin, and homologs thereof. A homolog of the present invention include those structurally related molecules that share at least 30% to about 99% structural similarity (or homology) with the parent composition. In one aspect, the homolog shares between 30% to 50% structural similarity with the parent composition. In another aspect, the homolog shares between 50% to 70% structural similarity with the parent composition. In yet another aspect, the homolog shares between 70% to 99% structural similarity with the parent composition. Preferably, the homolog of the present invention also possesses a similar functional property demonstrated by the parent. For example, if the parent demonstrates anti-microbial behavior towards particular microbes, then the homolog should possess similar activity, including having attenuated or greater anti-microbial activity.

In one aspect of the present invention, the chemical structure of prodigiosin comprises the following:

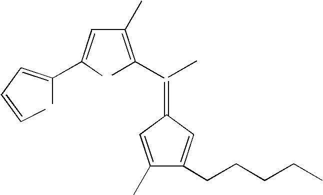

In another aspect of the present invention, the chemical structure of magnesidin comprises the following:

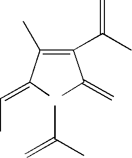

In this particular aspect, there is a homolog of magnesidin referred to as the C6 magnesidin and another homolog referred to as C4 magnesidin, their respective chemical structures are

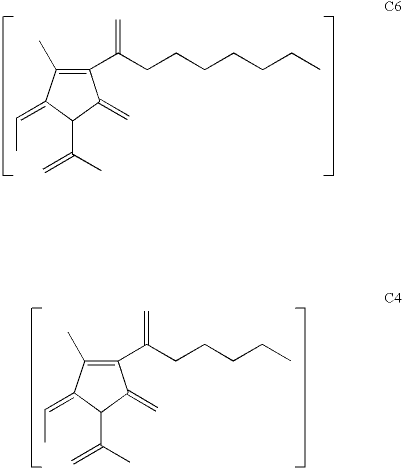

It should be understood that any modification to the compositions of the present invention are considered to be within the scope of this invention. Such modifications can include, but are not limited to, the addition of one or more groups, the elimination of one or more groups, for example, modifying the C6 magnesidin by eliminating one or more carbons along its alkyl group. Alternatively, the addition of groups such as one or more halogens, etc. to a structure is considered to be within the scope of the present invention. The important feature to any modification is that the modified structure must retain the anti-microbial activity of the parent. Again, this activity can be enhanced or attenuated due to the modification and still be considered to be within the scope of this invention.

The compositions of the present invention can be isolated from marine microbes using supercritical fluid technology (SCF). In one embodiment, the marine microbe is Vibrio gazogenes. The microbial cell mass obtained from one or more microbial cultures can be harvested by centrifugation at about 10,000×g for about 15 min, lyophilized and divided into two aliquots, one for organic extraction and the other for an aqueous fraction and supercritical fluids (SCF) fractionation of the cell pellet. The aliquots can be stored at −80° C. until further use.

Organic solvent extraction can be carried out on one half of the fermentation broth (250 mL) using conventional methods well known to those skilled in the art. Typically, 250 mL of the grown culture is extracted by adding about 125 mL butanol to a 500 mL Erlenmeyer flask. The flasks are shaken at around 250 rpm for approximately 30 min, and then allowed to stand for about 30 min. Most of the lower aqueous layer can be removed using a 1 mL plastic pipette attached to a vacuum pump. The flask contents can be transferred to centrifuge tubes that are centrifuged in a centrifuge at approximately 8,000 g for about 10 min to completely separate the phases. The upper butanol phase can then be collected by aspiration using a disposable Pasteur pipette, and transferred to a polypropylene or glass storage tubes (e.g., a 15 mL tube).

The other half or the fermentation broth (typically 250 mL) can be used for an aqueous fraction, and SCF fractionation of the cell pellet. This second aliquot can be centrifuged at about 8,000×g to collect the cell pellet, which is then dried. SCF fractionations are carried out on an ISCO (Lincoln, Nebr.) SFX 3560 automated extractor, see FIG. 1.

As shown in FIG. 2, a fluid becomes supercritical at conditions that equal or exceed both its critical temperature and critical pressure. Carbon dioxide, for example, becomes a supercritical fluid at conditions that equal or exceed its critical temperature of 31.1° C. and its critical pressure of 1,070 psig. In the supercritical fluid region, normally gaseous substances such as carbon dioxide become dense phase fluids, which have enhanced thermodynamic properties of solvation, selection and expansion. Utilizing small quantities of polar entrainers such as alcohol can readily modify selectivity. Near-critical fluids will have properties that are similar to supercritical fluids.

The SCF process produces a unique spectrum of secondary metabolites, reduces interference from nuisance compounds and minimizes background noise in sensitive molecular assays. This process thus enhances the drug discovery process.

Returning to FIG. 1, the pumps of the system are independently controllable, allowing easy adjustment of the fluid composition. The dried cell pellet can be transferred to a 10 mL ISCO extraction cartridge 3, after which the cartridge can be filled with 3 mm diameter glass beads to reduce the dead volume. After loading a cartridge on the cartridge holder, the fractionation procedure is commenced. The system is brought to 3,000 psig and 40° C., and extracted for 10 min with pure CO₂. This fraction can then be collected in methanol in a glass vial 4. Next, rapid depressurization is carried out in order to disrupt the cells. The fractionation parameters can be set to: SFS CO₂ at 3,000 psig and extraction temperature 40° C., step extractions with methanol as cosolvent at 0, 5, 10, 20, and 50 vol %, each step being 10 min. Because some void volume may remain between the glass beads, the composition of the extraction medium did not change sharply or immediately when modifier flowrate is adjusted to give a new fluid composition. Each sample can yield multiple fractions, which can then be collected in methanol using a separate glass vials. The different collection vials are mounted in a carousel 5. The aqueous, butanol, ethyl acetate and SFS fractions can be tested for antiplaque bioactivity. Ethyl acetate tends to be more efficient than butanol in extracting the active ingredient form the fermentation broth and cells.

Dried aqueous and butanol extracts of samples from the above-described procedures can be mixed and run on a silica gel column and eluted using a hexane:ethyl acetate:methanol gradient. Multiple fractions can be collected from the silica gel column, and monitored by TLC on silica gel using, for example, hexane:acetone:methanol (2:1:0.1 v/v/v/) as the mobile phase. Similar fractions can be combined and run on a small C₁₈ column with a mobile phase of, for example, methanol:H₂O (85:15 v/v). Multiple fractions can be collected and monitored by TLC on, for example, silica gel using, for example, hexane:acetone:methanol (2:1:0.1 v/v/v/) as the mobile phase.

Approximately 0.20 mg each of the isolated fractions can be subjected to mass spectral analysis. Fast atom bombardment analysis can be carried out using a M-Scan's VG Analytical ZAB 2-SE high field mass spectrometer. A cesium ion gun can be used to generate ions for the acquired mass spectra that were recorded using, for example, a PDP-11 250J data system. Mass calibration can be performed using cesium iodide.

Any remaining isolated fractions can be subjected to NMR analysis. Fractions can be dried and dissolved in approximately 800 μL of CDCl₃ or DMSO-d₆ and subjected to analysis using a 400 MHz ¹H NMR spectrometer. Identification of chemical structures can be accomplished by interpretation of spectral data, primarily ¹H and COSY NMR spectroscopy.

An isocratic HPLC technique can be developed for characterizing any active substances found, for example, magnesidin and its homologs. HPLC assays can be conducted on, for example, a Suplecon C₁₈ column (5 μm, 4.6×150 mm) with a pre-column cartridge @ 30° C. An isocratic mobile phase of, for example, methanol: H₂O:trifluoroacetic acid (50:50:0.002 v/v) adjusted to maintain the pH above 2.0 can be used for analysis of the C₁₈ column fractions and standards. A mobile phase of, for example, methanol:H₂O:trifluoroacetic acid (50:50:0.0005 v/v) can be used for analysis of standards and samples generated from the separation procedure. The flowrate can be set to 1.0 mL/min and absorbance can be monitored continuously by a detector, for example, a photodiode array detector from 200 to 395 nm with chromatographic scans measurements made at 257 nm. An HPLC system that can be utilized is the Waters HPLC system equipped with a 600 Multisolvent Delivery System, 717 Autosampler, 996 Photodiode Array Detector and Millennium Chromatography Manager Software.

Isolated fractions containing the putative anti-microbial agent can be tested for its anit-microbial activity. Cultures of Actinomyces viscosus, Streptococcus mutans, and Actinobacillus actinomycete-mcomitans can be obtained from the American Type Culture Collection (ATCC) following instructions provided by ATCC. Frozen stocks of each bacteria can be prepared by adding 10% (v/v) volume of glycerol to an overnight culture; aliquots of the resulting suspension were then frozen at −80° C.

Minimum inhibitory concentrations (MIC) can be determined to identify the minimum concentration of a composition needed to inhibit the growth of the target organism using methods well known to those skilled in the art. Minimum bactericidal concentrations (MBC) can be determined to identify the minimum concentration of a composition needed to kill the target organism using methods well known to those skilled in the art. The MBC can be determined from the same plates set up for the MIC.

A biofilm assay can be conducted to assess the bactericidal activity of the target compositions when the challenge organisms are contained within a biofilm. The semi-purified Vibrio gazogenes extract can be assayed for bactericidal activity against orally-relevant microbes contained in a biofilm grown on a solid support.

The present invention can take on the form of an oral care/health care products known in the art. For example in one embodiment, one or more of the compositions of the present invention are contained in a dentifrice. The dentifrice is used in the normal manner on the teeth. Another embodiment comprises a solution of marine anti-microbial composition that is separate from a dentifrice. After dispensing the dentifrice onto a brush, the composition is dispensed onto the dentifrice and then mixed through the process of application to the teeth.

As a consequence of the present invention's adaptability to forms and packaging, a number of pharmaceutically acceptable excipients can be used in addition to the marine anti-microbial compositions of the present invention. Many of these excipients are those routinely known for use in the art. A fairly broad, but, incomplete list of these excipients is disclosed in U.S. Pat. No. 5,281,412 to Lukacovic et al., the teaching of which is incorporated in its entirety herein by reference.

By “pharmaceutically-acceptable excipient” or “pharmaceutically-acceptable oral carrier,” as used herein, it is meant one or more compatible solid or liquid filler diluents or encapsulating substances which are suitable for topical, oral administration. By “compatible,” as used herein, it is meant that the components of the composition are capable of being commingled without interaction in a manner which would substantially reduce the composition's stability and/or efficacy for treating or preventing dental plaque and diseases associated therewith according to the compositions and methods of the present invention.

The carriers or excipients of the present invention can include the usual and conventional components of tooth pastes (including gels and gels for subgingival application), mouth rinses, mouth sprays, chewing gums, and lozenges (including breath mints) as more fully described hereinafter.

The compositions of the present invention can be multi phase compositions or single phase compositions. Normally, each phase in a multi phase composition is in a separate container or in a single container with more than one chamber. In one aspect, prior to use of a multi phase composition by a consumer, the different phases are combined by co-extrusion of the separate phases, preferably at a 1:1 volume to volume ratio, and the composition is preferably used immediately after preparation, i.e. within about 5 minutes.

The multi phases, however, can be combined from about 1 minute to about 1 hour before use, or during the use of the composition. Multi phase containers are disclosed in U.S. Pat. No. 5,052,590 to Ratcliff and U.S. Pat. No. 4,330,531 to Alliger, the entire teaching of which is incorporated herein by reference.

The choice of a carrier to be used is basically determined by the way the composition is to be introduced into the oral cavity. If a tooth paste (including tooth gels, etc.) is to be used, then a “tooth paste carrier” is chosen as disclosed in, e.g., U.S. Pat. No. 3,988,433 to Benedict, the entire teaching of which is incorporated herein by reference (e.g., abrasive materials, sudsing agents, binders, humectants, flavoring and sweetening agents, etc.). If a mouth rinse is to be used, then a “mouth rinse carrier” is chosen, as disclosed in, e.g., U.S. Pat. No. '433 (e.g., water, flavoring and sweetening agents, etc.). Similarly, if a mouth spray is to be used, then a “mouth spray carrier” is chosen or if a lozenge is to be used, then a “lozenge carrier” is chosen (e.g., a candy base), candy bases being disclosed in, e.g., U.S. Pat. No. 4,083,955 to Grabenstetter et al., the entire teaching of which is incorporated herein by reference; if a chewing gum is to be used, then a “chewing gum carrier” is chosen, as disclosed in, e.g., U.S. Pat. No. '955 to (e.g., gum base, flavoring and sweetening agents). If a sachet is to be used, then a “sachet carrier” is chosen (e.g., sachet bag, flavoring and sweetening agents). If a subgingival gel is to be used (for delivery of actives into the periodontal pockets or around the periodontal pockets), then a “subgingival gel carrier” is chosen as disclosed in, e.g., U.S. Pat. No. 5,198,220 to Damani, and U.S. Pat. No. 5,242,910 to Damani, the entire teaching of which is incorporated herein by reference. Carriers suitable for the preparation of compositions of the present invention are well known in the art. Their selection will depend on secondary considerations like taste, cost, and shelf stability, etc.

Some preferred compositions of the subject invention are in the form of dentifrices, such as tooth pastes, tooth gels and tooth powders. Components of such tooth paste and tooth gels generally include one or more of the following: a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such tooth paste or tooth gel can also include one or more of the following: an anticaries agent (from about 0.05% to about 0.3% as fluoride ion), and an anti-calculus agent (from about 0.1% to about 13%). Tooth powders, of course, contain substantially all non-liquid components.

Other preferred compositions of the present invention are non-abrasive gels, including subgingival gels, which generally include a thickening agent (from about 0.1% to about 20%), a humectant (from about 0.1% to about 90%), a flavoring agent (from about 0.04% to about 2%), a sweetening agent (from about 0.1% to about 3%), a coloring agent (from about 0.01% to about 0.5%), water (from about 2% to about 45%), and can comprise an anticaries agent (from about 0.05% to about 0.3% as fluoride ion), and an anti-calculus agent (from about 0.1% to about 13%).

Other preferred compositions of the subject invention are mouth washes, including mouth sprays. Components of such mouth washes and mouth sprays typically include one or more of the following: water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of the following: an anticaries agent (from about 0.05% to about 0.3% as fluoride ion), and an anti-calculus agent (from about 0.1% to about 3%).

Other preferred compositions of the subject invention are dental solutions. Components of such dental solutions generally include one or more of the following: water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Chewing gum compositions typically include one or more of the following: a gum base (from about 50% to about 99%), a flavoring agent (from about 0.4% to about 2%) and a sweetening agent (from about 0.01% to about 20%).

The term “lozenge” as used herein includes: breath mints, troches, pastilles, microcapsules, and fast-dissolving solid forms including freeze dried forms (cakes, wafers, thin films, tablets) and fast-dissolving solid forms including compressed tablets. The term “fast-dissolving solid form” as used herein means that the solid dosage form dissolves in less than about 60 seconds, preferably less than about 15 seconds, more preferably less than about 5 seconds, after placing the solid dosage form in the oral cavity. Fast-dissolving solid forms are disclosed in U.S. Pat. No. 4,642,903; U.S. Pat. No. 4,946,684; U.S. Pat. No. 4,305,502; U.S. Pat. No. 4,371,516; U.S. Pat. No. 5,188,825; U.S. Pat. No. 5,215,756; U.S. Pat. No. 5,298,261; U.S. Pat. No. 3,882,228; U.S. Pat. No. 4,687,662; U.S. Pat. No. 4,642,903, the entire teachings of which are incorporated herein by reference.

Lozenges include discoid-shaped solids comprising a therapeutic agent in a flavored base. The base can be a hard sugar candy, glycerinated gelatin or combination of sugar with sufficient mucilage to give it form. These dosage forms are generally described in Remington: The Science and Practice of Pharmacy, 19.sup.th Ed., Vol. II, Chapter 92, 1995, the entire teaching of which is incorporated herein by reference. Lozenge compositions (compressed tablet type) typically include one or more fillers (compressible sugar), flavoring agents, and lubricants. Microcapsules of the type contemplated herein are disclosed in U.S. Pat. No. 5,370,864 to Peterson et al., the entire teaching of which is herein incorporated by reference.

Types of carriers or oral care excipients which can be included in compositions of the present invention, along with specific non-limiting examples, are set forth in following sections.

Dental abrasives useful in the topical, oral carriers of the compositions of the instant invention include many different materials. The material selected must be one which is compatible within the composition of interest and does not excessively abrade dentin. Suitable abrasives include, but not limited to, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 to Cooley and Grabenstetter, the entire teaching of which is incorporated herein by reference. Suitable resins include, but limited to, melamines, phenolics, ureas, melamine-ureas, melamine-formaldehydes, urea-formaldehyde, melamine-urea-formaldehydes, cross-linked epoxides, and cross-linked polyesters. Mixtures of abrasives can also be used.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, and DiGiulio, U.S. Pat. No. 3,862,307, the entire teaching of which is incorporated herein by reference. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, Zeodent®, particularly the silica carrying the designation Zeodent 119®. The types of silica dental abrasives useful in the tooth pastes of the present invention are described in more detail in Wason, U.S. Pat. No. 4,340,583, the entire teaching of which is incorporated herein by reference. The abrasive in the tooth paste compositions described herein is generally present at a level of from about 6% to about 70% by weight of the composition. Preferably, tooth pastes contain from about 10% to about 50% of abrasive, by weight of the composition.

A preferred precipitated silica is the silica disclosed in U.S. Pat. No. 5,603,920, U.S. Pat. No. 5,589,160, U.S. Pat. No. 5,658,553, and U.S. Pat. No. 5,651,958, the teachings of which are incorporated herein by reference in their entirety.

Mixtures of abrasives can be used. The total amount of abrasive in dentifrice compositions of the subject invention preferably range from about 6% to about 70% by weight; tooth pastes preferably contain from about 10% to about 50% of abrasives, by weight of the composition. Solution, mouth spray, mouthwash and non-abrasive gel compositions of the subject invention typically contain no abrasive.

Suitable sudsing agents are those which are reasonably stable and form foam throughout a wide pH range. Sudsing agents include nonionic, anionic, amphoteric, cationic, zwitterionic, synthetic detergents, and mixtures thereof. Many suitable nonionic and amphoteric surfactants are disclosed by U.S. Pat. No. 3,988,433, and U.S. Pat. No. 4,051,234, and many suitable nonionic surfactants are disclosed by Agricola et al., U.S. Pat. No. 3,959,458, the teachings of which are incorporated herein in their entirety by reference.

Nonionic surfactants which can be used in the compositions of the present invention can be broadly defined as compounds produced by the condensation of alkylene oxide groups (hydrophilic in nature) with an organic hydrophobic compound which can be aliphatic or alkyl-aromatic in nature. Examples of suitable nonionic surfactants include poloxamers (sold under trade name Pluronic), polyoxyethylene sorbitan esters (sold under trade name “Tween”), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials.

The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed.

The present composition can typically comprise a nonionic, amphoteric, or combination of nonionic and amphoteric surfactant each at a level of from about 0.025% to about 5%, preferably from about 0.05% to about 4%, and most preferably from about 0.1% to about 3%.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. The present composition typically comprises an anionic surfactant at a level of from about 0.025% to about 9%, preferably from about 0.05% to about 7%, and most preferably from about 0.1% to about 5%.

The present invention can also incorporate free fluoride ions. Preferred free fluoride ions can be provided by sodium fluoride, stannous fluoride, indium fluoride, and sodium monofluorophosphate. Sodium fluoride is the most preferred free fluoride ion. Norris et al., U.S. Pat. No. 2,946,725 and Widder et al., U.S. Pat. No. 3,678,154, the entire teaching of which are incorporated herein by reference, disclose such salts as well as others.

The present composition can contain from about 50 ppm to about 3500 ppm, and preferably from about 500 ppm to about 3000 ppm of free fluoride ions.

In preparing tooth paste or gels, it is necessary to add some thickening material to provide a desirable consistency of the composition. Preferred thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopolt series. Particularly preferred carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. No. 5,198,220 to Damani, U.S. Pat. No. 5,242,910 to Damani, and U.S. Pat. No. 4,443,430 to Mattei, the entire teachings of which all are incorporated herein by reference.

Thickening agents in an amount from about 0.1% to about 15%, preferably from about 2% to about 10%, more preferably from about 4% to about 8%, by weight of the total tooth paste or gel composition, can be used. Higher concentrations can be used for chewing gums, lozenges (including breath mints), sachets, non-abrasive gels and subgingival gels.

As discussed above, the polyalcohols of the present invention can also act as a humectant. Humectants serve to keep tooth paste compositions from hardening upon exposure to air, to give compositions a moist feel to the mouth, and, for particular humectants, to impart desirable sweetness of flavor to tooth paste compositions. Suitable humectants include glycerin, sorbitol, butylene glycol, polyethylene glycol, and especially sorbitol and glycerin.

Flavoring agents can also be added to the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, α-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

As in the case of the humectants, sweetening agents that can be used in the present invention can include the non-cariogenic carbohydrates disclosed above. Sweeteners useful in compositions of the present invention include sucrose, glucose, saccharin, dextrose, levulose, lactose, mannitol, sorbitol, fructose, maltose, xylitol, saccharin salts, thaumatin, aspartame, D-tyyptophan, dihydrochalcones, acesulfame and cyclamate salts, especially sodium cyclamate and sodium saccharin, and mixtures thereof. A composition preferably contains from about 0.1% to about 10% of these agents, preferably from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, preferably from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425 to Amano et al., the entire teaching of which is incorporated herein by reference. WS-3 and other agents are described in U.S. Pat. No. 4,136,163 to Watson et al., the teachings are herein incorporated by reference in their entirety.

Preferred salivating agents of the present invention include Jambu® manufactured by Takasago. Preferred warming agents include capsicum and nicotinate esters, such as benzyl nicotinate. Preferred numbing agents include benzocaine, lidocaine, clove bud oil, and ethanol.

The present invention also includes an anti-calculus agent, preferably a pyrophosphate ion source which is from a pyrophosphate salt. The pyrophosphate salts useful in the present compositions include the dialkali metal pyrophosphate salts, tetraalkali metal pyrophosphate salts, and mixtures thereof. Disodium dihydrogen pyrophosphate (Na₂H₂ P₂O₇), tetrasodium pyrophosphate (Na₄ P₂O₇), and tetrapotassium pyrophosphate (K₄ P₂O₇) in their unhydrated as well as hydrated forms are the preferred species. In compositions of the present invention, the pyrophosphate salt can be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions can be from about 1% to about 15%, preferably from about 1.5% to about 10%, and most preferably from about 2% to about 6%. Free pyrophosphate ions can be present in a variety of protonated states depending on a the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate can be the anhydrous salt form or the decahydrate form, or any other species stable in solid form in the dentifrice compositions. The salt is in its solid particle form, which can be its crystalline and/or amorphous state, with the particle size of the salt preferably being small enough to be aesthetically acceptable and readily soluble during use. The amount of pyrophosphate salt useful in making these compositions is any tartar control effective amount, and is generally from about 1.5% to about 15%, preferably from about 2% to about 10%, and most preferably from about 3% to about 8%, by weight of the dentifrice composition.

Compositions can also comprise a mixture of dissolved and undissolved pyrophosphate salts. Any of the above mentioned pyrophosphate salts can be used. The pyrophosphate salts are described in more detail in Kirk & Othmer, Encyclopedia of Chemical Technology, Third Edition, Volume 17, Wiley-Interscience Publishers (1982), the teaching of which is incorporated herein by reference in its entirety.

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the teaching of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

The present invention can also include an alkali metal bicarbonate salt. Alkali metal bicarbonate salts are soluble in water and unless stabilized, tend to release carbon dioxide in an aqueous system. Sodium bicarbonate, also known as baking soda, is the preferred alkali metal bicarbonate salt. The present composition can contain from about 0.5% to about 30%, preferably from about 0.5% to about 15%, and most preferably from about 0.5% to about 5% of an alkali metal bicarbonate salt.

Water employed in the preparation of commercially suitable oral compositions should preferably be of low ion content and free of organic impurities. Water generally comprises from about 5% to about 70%, and preferably from about 20% to about 50%, by weight of the composition herein. These amounts of water include the free water which is added plus that which is introduced with other materials, such as with sorbitol.

Titanium dioxide can also be added to the present composition. Titanium dioxide is a white powder which adds opacity to the compositions. Titanium dioxide generally comprises from about 0.25% to about 5% by weight of the dentifrice compositions.

Antimicrobial anti-plaque agents can also by optionally present in oral compositions. Such agents can include, but are not limited to, triclosan, 5-chloro-2-(2,4-dichlorophenoxy)-phenol, as described in The Merck Index, 11th ed. (1989), pp. 1529 (entry no. 9573), U.S. Pat. No. 3,506,720, and in European Patent Application No. 0,251,591; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minocycline, and metronidazole; and analogs and salts of the above antimicrobial antiplaque agents. If present, the antimicrobial antiplaque agents generally comprise from about 0.1% to about 5% by weight of the compositions of the present invention.

Anti-inflammatory agents can also be present in the oral compositions of the present invention. Such agents can include, but are not limited to, non-steroidal anti-inflamnnatory agents such as aspirin, ketorolac, flurbiprofen, ibuprofen, naproxen, indomethacin, aspirin, ketoprofen, piroxicam and meclofenarnic acid, and mixtures thereof. If present, the anti-inflammatory agents generally comprise from about 0.001% to about 5% by weight of the compositions of the present invention. Ketorolac is described in U.S. Pat. No. 5,626,838, the entire teaching of which is herein incorporated by reference.

Other optional agents include synthetic anionic polymeric polycarboxylates being employed in the form of their free acids or partially or preferably fully neutralized water soluble alkali metal (e.g., potassium and preferably sodium) or ammonium salts and are disclosed in U.S. Pat. No. 4,152,420 to Gaffar, U.S. Pat. No. 3,956,480 to Dichter et al., U.S. Pat. No. 4,138,477 to Gaffar, U.S. Pat. No. 4,183,914 to Gaffar et al., and U.S. Pat. No. 4,906,456 to Gaffar et al., the entire teachings of which are herein incorporated by reference. Preferred are 1:4 to 4:1 copolymers of maleic anhydride or acid with another polymerizable ethylenically unsaturated monomer, preferably methyl vinyl ether (methoxyethylene) having a molecular weight (MW) of about 30,000 to about 1,000,000. These copolymers are available, for example, as Gantrez AN 139 (MW 500,000), A.N. 119 (MW 250,000) and preferably S-97 Pharmaceutical Grade (MW 70,000), of GAF Corporation.

The present invention can also optionally comprise selective H-2 antagonists including compounds disclosed in U.S. Pat. No. 5,294,433 to Singer et al., the entire teaching of which is herein incorporated by reference.

A safe and effective amount of the compositions of the present invention can be topically applied to the mucosal tissue of the oral cavity, to the gingival tissue of the oral cavity, and/or to the surface of the teeth, for the treatment or prevention of the above mentioned diseases or conditions of the oral cavity, in several conventional ways. For example, the gingival or mucosal tissue may be rinsed with a solution (e.g., mouth rinse, mouth spray), a dentifrice (e.g., tooth paste, tooth gel or tooth powder), and the gingival/mucosal tissue or teeth are bathed in the liquid and/or lather generated by brushing the teeth. Other non-limiting examples include applying a non-abrasive gel or paste directly to the gingival/mucosal tissue or to the teeth with or without an oral care appliance described below; a chewing gum; or by chewing or sucking on a lozenge.

Preferred methods of applying the composition to the gingival/mucosal tissue and/or the teeth are via rinsing with a mouth rinse solution and via brushing with a ?0.4 dentifrice. Other methods of topically applying the composition to the gingival/mucosal tissue and the surfaces of the teeth are apparent to those skilled in the art.

For the method of preventing and treating diseases or conditions of the oral cavity of the present invention, the composition is preferably applied to the gingival/mucosal tissue and/or the teeth (e.g., by rinsing with a mouth rinse, directly applying a non-abrasive gel with or without a device, applying a dentifrice or a tooth gel with a toothbrush, sucking or chewing a lozenge preferably for at least about 10 seconds, preferably from about 20 seconds to about 10 minutes, more preferably from about 30 seconds to about 60 seconds). The method often involves expectoration of most of the composition following such contact. The frequency of such contact is preferably from about once per week to about four times per day, more preferably from about thrice per week to about three times per day, even more preferably from about once per day to about twice per day. The period of such treatment typically ranges from about one day to a lifetime. For particular oral care diseases or conditions the duration of treatment depends on the severity of the oral disease or condition being treated, the particular delivery form utilized, and the patient's response to treatment. If delivery to the periodontal pockets is desirable, such as with the treatment of periodontal disease, a mouth rinse can be delivered to the periodontal pocket using a syringe or water injection device. These devices are known to one skilled in the art. Devices of this type include Water Pik® by Teledyne Corporation. After irrigating, the subject can swish the rinse in the mouth to also cover the dorsal tongue and other gingival and mucosal surfaces. In addition a tooth paste, non-abrasive gel, toothgel, etc. can be brushed onto the tongue surface and other gingival and mucosal tissues of the oral cavity.

### EXAMPLE

(A) Marine Microorganism Fermentation Media

A marine organism sample, Vibrio gazogenes, was maintained as a frozen stock culture prepared in 10% glycerol and stored at −80° C. Prior to initiating liquid cultures the stock culture was streaked on saltwater agar and examined by colony characteristics, Gram-staining, and cell morphology to ensure purity.

The media used for fermentation was prepared in an artificial seawater (ASW) base. The advantage of using ASW is that its chemical composition remains consistent, as compared with natural seawater. GP2 formulation ASW (Bidwell et al., 1985; and Spotte and et al., 1984, the entire teaching of which are incorporated herein by reference) was used for all fermentations. GP2 was chosen because it has the closest chemical composition to natural seawater and, since it is prepared as two solutions that are mixed after autoclaving, it does not precipitate. This allows for use of full-strength ASW for marine fermentations. The composition of the GP2 artificial sea water is listed in Table 1.

TABLE 1
Composition of GP2 Artificial Seawater (per Liter)

| | Amount | | Amount |
|---|---|---|---|
| Chemical | (grams) | Chemical | (grams) |
| NaCl | 23.9 | Na₂SO₄ | 4 |
| Kcl | 0.698 | NaHCO₃ | 0.193 |
| Kbr | 0.1 | Na₂B₄O₇.10H₂O | 0.039 |
| MgCl₂.6H₂O | 0.108 | CaCl₂.2H₂O | 1.5 |
| SrCl₂.6H₂O | 0.0243 | NaH₂PO₄.H₂O | 0.0128 |
| Ferric citrate.H₂O | 2.42 × 10⁻⁵ | Na₂MoO₄.2H₂O | 8.3 × 10⁻⁵ |
| KI | 2.18 × 10⁻⁵ | ZnSO₄.7H₂O | 2.18 × 10⁻⁵ |
| NaVO₃ | 6.1 × 10⁻⁶ | MnSO₄.H₂O | 6.08 × 10⁻⁷ |
| Urea | 4.47 × 10⁻² | Thiamine.HCl | 1.95 × 10⁻³ |
| Biotin | 9.99 × 10⁻⁷ | Cyanocobalamine | 9.77 × 10⁻⁷ |

Cultures for routine use were maintained on the appropriate agar slants or plates kept at 4° C. Cultures were routinely examined by colony characteristics, Gram-staining, and cell morphology. Four liquid fermentation media differing in chemical composition were used to grow any given culture. Use of more than one medium helps to maximize the diversity of secondary metabolites. The media described below are high or low in particular nutrients, specifically carbon and nitrogen. Carbon sources were glucose, glycerol, or sodium acetate. Nitrogen sources were peptone, yeast extract, and beef extract. The following protocol for the four medium compositions was used: (a) HCLN: 0.5% glucose, 0.5% glycerol, 0.2% peptone, and 0.2% yeast extract[Media R]; (b) LCHN: 0.2% glucose, 0.2% glycerol, 0.1% Na acetate, 0.8% peptone, 0.2% yeast extract[Media S]; (c) LCLN: 0.2% glucose, 0.2% peptone, 0.2% beef extract[Media T]; and (d) HCHN: 0.5% glucose, 0.5% glycerol, 0.2% Na acetate, 0.8% peptone, and 0.2% beef extract[Media U]. Marine fermentation media were prepared in GP2 artificial seawater per the listing in Table 1 above.

(1) Initial Growth of Sample

Isolated colonies were used to initiate starter cultures of 5 mL of each of four marine media R, S, T, and U prepared in 10 mL flasks. Each flask was sterilized by autoclaving at 121° C. and 1.1 kPa for 15 min. Each flask was incubated for 4 days at 37° C. on a shaker at 250 rpm (2.5 cm stroke), to provide adequate oxygen levels during the growth period. These starter cultures were used to inoculate the four 250 mL volumes of each media type in one-liter flasks. The flasks were incubated at 25° C. on a shaker 250 rpm (2.5 cm stroke) for 7 days. The 7-day incubation period ensured that the culture was in stationary phase of growth, where most of the secondary metabolite production is expected to take place. At the end of the seven-day incubation period, the cultures grown in the same media type were combined into two 500 mL batches. Fermentations were carried out in one-liter Erlenmeyer flasks to provide adequate biomass for isolation of the active compound. Each flask contained 500 mL of the appropriate medium, and was sterilized for 15 min. One batch was utilized for antimicrobial screening. The second 500 mL fraction of each culture was split and extracted as described in the next section.

(2) Large-Scale Fermentation of the Sample Marine Microorganism

The marine isolate was grown under optimized fermentation conditions in 10-liter quantities to provide adequate biomass for isolation of the active compound. The sample starter culture was used to inoculate the ten-liter fermentor. The fermentor was incubated at 30° C. with 100-150 rpm agitation and 1,500 cc/min air input for three days.

Although this temperature is above that found in the marine environment, the incubation temperature of 30° C. provided optimal growth of this organism. Growth curves of this organism demonstrate that this culture reaches stationary phase within 24 hours, and there is no difference in the activity in the cell pellet after 24 hours. A 3-day incubation period ensured that the culture was in stationary phase of growth, where most of the secondary metabolite production is expected to take place. At the end of the 3-day incubation period, the microbial cell mass was harvested by centrifugation at 10,000×g for 15 min, lyophilized and divided into two aliquots, one for organic extraction and the other for an aqueous fraction and supercritical fluids (SCF) fractionation of the cell pellet. The aliquots were stored at −80° C. until further use. A total of fourteen (14) large-scale (10-liter) fermentation runs was made.

(B) Fractionation of the Marine Microorganism Sample

Organic solvent extraction was carried out on one half of the fermentation broth (250 mL) using conventional methods utilized in the pharmaceutical industry. Typically, 250 mL of the grown culture was extracted by adding 125 ML butanol to the 500 mL Erlenmeyer flask. The flasks were shaken at 250 rpm for 30 min, and were then allowed to stand for 30 min. Most of the lower aqueous layer was suctioned off with a 1 mL plastic pipette attached to a vacuum pump. The flask contents were transferred to centrifuge tubes that were centrifuged in a Sorvall RC2-B centrifuge at 8,000 g for 10 min to completely separate the phases. The upper butanol phase was collected by aspiration using a disposable Pasteur pipette, and transferred to a 15 mL polypropylene or glass storage tubes.

The other half or the fermentation broth (typically 250 mL) was used for an aqueous fraction, and SCF fractionation of the cell pellet. The second aliquot was centrifuged at 8,000×g to collect the cell pellet, which was then dried. SCF fractionations were carried out on an ISCO (Lincoln, Nebr.) SFX 3560 automated extractor. As shown in FIG. 1, this is a dual pump system, utilizing syringe pump 1 for neat critical fluid and syringe pump 2 for modifier.

The pumps are independently controllable, allowing easy adjustment of the fluid composition. The dried cell pellet was transferred to a 10 ML ISCO extraction cartridge 3, after which the cartridge was filled with 3 mm diameter glass beads to reduce the dead volume. After loading a cartridge on the cartridge holder, the fractionation procedure was commenced. The system was brought to 3,000 psig and 40° C., and extracted for 10 min with pure CO₂. This fraction was collected in methanol in a glass vial 4. Next, rapid depressurization was carried out in order to disrupt the cells. Next, the fractionation parameters were set to: SFS CO₂ at 3,000 psig and extraction temperature 40° C., step extractions with methanol as cosolvent at 0, 5, 10, 20, and 50 vol %, each step being 10 min. Because some void volume remained between the glass beads, the composition of the extraction medium did not change sharply or immediately when modifier flowrate was adjusted to give a new fluid composition. Each sample thus yielded 6 fractions, which were collected in methanol in separate glass vials. The different collection vials are mounted in a carousel 5. The aqueous, butanol, ethyl acetate and SFS fractions were tested for anti-plaque bioactivity.

Ethyl acetate was much more efficient than butanol in extracting the active ingredient form the fermentation broth and cells. In the SCF fractionation of the cell pellet, the most active fractions were obtained with SFS CO₂ with 20 vol % methanol as cosolvent at 3,000 psig and 40° C. This fraction had a MIC of <0.97 μg/mL against A. viscosus.

(C) Isolation, Purification and Characterization of Magnesidin

The two anti-microbial compounds of the marine microorganism, Vibrio gazogenes, as identified by fatty acid-GC analysis, were isolated by a combination of Thin Layer Chromatography and Low Pressure Column Chromatography utilizing silica and C₁₈ columns. Mass spectra and proton NMR were utilized to identify these compounds, as magnesidin and prodigiosin—a red compound that has both antimicrobial and antifungal activity. Magnesidin was further characterized by its UV spectra and HPLC analyses.

(1) Column Chromatography

Dried aqueous and butanol extracts of samples labeled APP214R, APP214S, APP214T and APP214U (50 mg total) were mixed and run on a silica gel column and eluted with hexane:ethyl acetate:methanol gradient. Twenty-three fractions were collected from the silica gel column, and were monitored by TLC on silica gel F254 with hexane:acetone:methanol (2:1:0.1 v/v/v/) as the mobile phase. Similar fractions were combined and were run on a small C₁₈ column with a mobile phase of methanol:H₂O (85:15 v/v). Thirty 20 mL fractions were collected and monitored by TLC on silica gel F254 with hexane:acetone:methanol (2:1:0.1 v/v/v/) as the mobile phase. Similar fractions were combined to provide a total of five fractions.

Fraction 1 contained four compounds (B 1-B4) in addition to a red compound. Fraction 2 contained compounds B2, B3, and B4. Fractions 3-5 contain low levels of other compounds. Fraction 1 was dried onto C₁₈ and eluted with a mobile phase of methanol:water (85:15 v/v). Thirty 20 mL fractions were collected. The two bioactive compounds were collected in semi-purified form from fractions 23-25. The four compounds isolated were analyzed by NMR.

(2) Mass Spectral and NMR Analysis

Approximately 0.20 mg each of four isolated fractions were sent to M-Scan, Inc., West Chester, Pa. for mass spectral analysis. The fractions were: APP214RSTUAB1 (putatively a pure lipophilic compound), APP214RSTUAB2 (mixture of two major compounds, pink in color), APP214RSTUAB3 (could be relatively pure magnesidin) and APP214RSTUAB4 (could be relatively pure magnesidin). Fast atom bombardment analysis was carried out on M-Scan's VG Analytical ZAB 2-SE high field mass spectrometer. A cesium ion gun was used to generate ions for the acquired mass spectra that were recorded using a PDP-11 250J data system. Mass calibration was performed using cesium iodide.

The remainder of the four isolated fractions APP214RSTUAB1, APP214RSTUAB2, APP214RSTUAB3 and APP214RSTUAB4 were sent to Dr. D. John Faulkner, Scripps Institution of Oceanography, La Jolla, Calif. for NMR analysis. Fractions were dried and dissolved in 800 μL of CDCl₃ or DMSO-d₆ and analyzed using a 400 MHz ¹H NMR spectrometer. Identification of chemical structures was accomplished by interpretation of spectral data, primarily ¹H and COSY NMR spectroscopy.

Fraction APP214RSTUAB1 was identified by mass spectral and NMR analyses to be a lipophilic contaminant. Fraction APP214RSTUAB2 also contained this contaminant as the major constituent as indicated by the ¹H NMR spectrum and the MS peaks. A peak at m/z=324 is compatible with the [M+H]+ peak for prodigiosin, a red pigment commonly found in marine bacteria. This structure is supported by the NMR peaks at δ 2.4 (Me), 4.02 (Ome), 6.12 (pyrrole), 6.38 (pyrrole), 6.72 (pyrolle), 6.72 (pyrrole), 6.92 (pyrolle), 6.98 (C═CH), and 12.1-12.7 (NH). This pigment was identified as prodigiosin.

A mass spectral scan of APP214RSTUAB3 is shown in FIG. 3. The ions observed at m/z 609 and 924 are consistent with those for the [2M+Mg+H] and [3M+2Mg] ions respectively, found in magnesidin. Numerous other significant possible sample derived ions are observed as well. The ions observed at m/z 136, 154 and 307 can be assigned to the matrix. The mass spectrum is almost identical to the published spectrum of magnesidin and constitutes a positive identification for the C6 homolog. The ¹H and COSY NMR spectra, see FIGS. 4 & 5, confirm the assignment, but contain peaks that suggest the presence of an impurity that is either a magnesidin containing a different hydrocarbon chain or a fatty acid.

Mass spectral scans of APP214RSTUAB4 were inconclusive. The ¹H NMR suggests that this fraction is a lower homolog (C4) of magnesidin with two less methylene groups in the side chain. This fraction was estimated to be >95% pure.

(3) HPLC Analysis

An isocratic HPLC technique was developed for characterizing magnesidin and its homologs. HPLC assays were conducted on a Suplecon C₁₈ column (5 μm, 4.6×150 mm) with a pre-column cartridge @ 30° C. An isocratic mobile phase of methanol: H₂O:trifluoroacetic acid (50:50:0.002 v/v) adjusted to maintain the pH above 2.0 was used for analysis of the C₁₈ column fractions and the standards. A mobile phase of methanol:H₂O:trifluoroacetic acid (50:50:0.0005 v/v) was used for analysis of standards and the samples generated from the gross separation experiment. The flowrate was set to 1.0 mL/min and absorbance was monitored continuously by a photodiode array detector from 200 to 395 nm with chromatographic scans measurements made at 257 nm. The HPLC system utilized consisted of a Waters HPLC system equipped with a 600 Multisolvent Delivery System, 717 Autosampler, 996 Photodiode Array Detector and Millennium Chromatography Manager Software.

A standard curve of the lower (C4) homolog, shown in FIG. 3, was prepared by inoculating 10, 20, and 40 μL of the 1 mg/mL standard. The concentration was corrected based upon the NMR data that demonstrated this fraction was ˜95% pure. An HPLC chromatographic scan of the C4 magnesidin homolog is shown in FIG. 6.

A standard curve of the higher magnesidin homolog (C6) could not be prepared because the isolated compound was not sufficient pure and sufficient quantities were not available to do further purification by crystallization. A 40 μL injection was made and the area of the peak was used to give an estimate of the concentration of this homolog in each of the fractions. An HPLC chromatographic scan of the C6 magnesidin homolog is shown in FIG. 7.

(D) Biological Activities of Purified Active Components

Cultures of Actinomyces viscosus, Streptococcus mutans, and Actinobacillus actinomycete-mcomitans were obtained from the American Type Culture Collection (ATCC) following instructions provided by ATCC. Frozen stocks of each bacteria were prepared by adding 10% (v/v) volume of glycerol to an overnight culture; aliquots of the resulting suspension were then frozen at −80° C.

(1) Minimum Inhibitory and Minimum Bactericidal Activity

Minimum inhibitory concentrations (MIC) were determined to identify the minimum concentration of a compound needed to inhibit the growth of the target organism. Minimum bactericidal concentrations (MBC) were determined to identify the minimum concentration of a compound needed to kill the target organism. The MBC was determined from the same plates set up for the MIC.

The MIC and MBC of the semi-purified Vibrio gazogenes extract is given in Table 2. The MIC and MBC of the two purified homologs of magnesidin were tested against orally relevant microorganisms. The MIC and MBC values for the purified homologs are given in Tables 3 and 4, respectively. Triclosan and chlorhexidine were included as positive controls.

TABLE 2
MIC and MBC of Semi-Purified Vibrio gazogenes

| Challenge Microorganism | MIC (μg/mL) | MBC (μg/mL) |
|---|---|---|
| A. viscosus | <1.95 | <1.95 |
| A. actinomycetemcomitans | 125 | 125 |
| S. mutans | 15.6 | 125 |

TABLE 3
MIC of C4 and C6 Magnesidin Homologs

| | MIC (μg/mL) | | | |
|---|---|---|---|---|
| | Triclo- | Chlorhex- | C4 | C6 |
| Challenge Microorganism | san | idine | Homolog | Homolog |
| A. viscosus | 3.9 | 3.9 | 15.63 | <0.75 |
| A. actinomycetemcomitans | <0.75 | 3.9 | 62.5 | 62.5 |
| S. mutans | 7.8 | 1.95 | 125 | 3.9 |

TABLE 4
MBC of C4 and C6 Magnesidin Homologs

| | MBC (μg/mL) | | | |
|---|---|---|---|---|
| | Triclo- | Chlorhex- | C4 | C6 |
| Challenge Microorganism | san | idine | Homolog | Homolog |
| A. viscosus | 3.9 | 7.8 | 31.25 | <0.75 |
| A. actinomycetemcomitans | <0.75 | 3.9 | 125 | 125 |
| S. mutans | 15.6 | 1.95 | 62.5 | 7.8 |

(2) Biofilm Assay

A biofilm assay was conducted to access the bactericidal activity of the target compounds when the challenge organisms are contained within a biofilm. The semi-purified Vibrio gazogenes extract was assayed for bactericidal activity against orally-relevant microbes contained in a biofilm grown on a solid support. The data listed in Table 5 indicates that semi-purified Vibrio gazogenes compared favorably with the activity of the control agent triclosan when tested against A. actinomycetemcomitans and A. viscosus. The semi-purified material, at the tested concentrations, had no apparent bactericidal activity against S. mutans. This may or may not be significant since the concentration of the bioactive constituent of the semi-purified Vibrio gazogenes extract was not accurately determined and could have been significantly lower than estimated due to the presence of impurities.

TABLE 5
Microbial Biofilm Control by Semi-Purified APP-214 and Triclosan

| | Vibrio | Vibrio | | |
|---|---|---|---|---|
| | gazogenes¹ | gazogenes¹ | Triclosan¹ | Bacteria |
| Microorganism | (250 μg/mL) | (125 μg/mL) | (125 μg/mL) | Only² |
| A. viscosus | ND | 5.0 × 10⁶ | 6.0 × 10⁵ | 1.1 × 10⁸ |
| A. actino- | 5 × 10⁵ | 1.3 × 10⁶ | 2.1 × 10⁶ | 6.5 × 10⁸ |
| mycetemcomitans | | | | |
| S. mutans | 6.0 × 10⁷ | 4.0 × 10⁷ | 0 | 6.0 × 10⁷ |

¹1 hour incubation in test agent
²1 hour incubation in PBS

(E) Toxicity

Magnesidin is considered fairly non-toxic, the LD₅₀ in mice being 50 mg/kg (intraperitoneal) and 1,000 mg/kg (oral or subcutaneous), see Gandhi et al., 1973; and Nazareth et al., 1975, the teaching of which is incorporated herein by reference in its entirety.

While this invention has been particularly shown and described with references to specific embodiments, it will be understood by those skilled in the art that various changes in form and details may be made therein without departing from the spirit and scope of the invention as defined by the appended claims.

1. A composition for reducing dental plaques comprising a marine anti-microbial agent selected from the group consisting of prodigiosin, magnesidin and homologs thereof.

2. The composition of claim 1, wherein the structure of prodigiosin is the following:

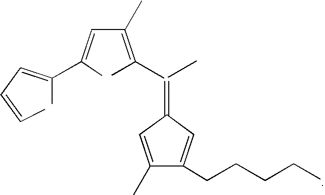

3. The composition of claim 1, wherein the structure for magnesidin is the following:

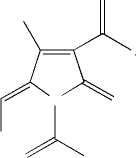

4. The composition of claim 1, wherein said homologs are from about 30% to about 99% structurally identical to its parent.

5. The composition of claim 1, wherein said homologs are from about 30% to about 50% structurally identical to its parent.

6. The composition of claim 1, wherein said homologs are from about 50% to about 70% structurally identical to its parent.

7. The composition of claim 1, wherein said homologs possess similar functional activity as demonstrated by the parent compound.

8. The composition of claim 7, wherein said functional activity comprises anti-microbial activity.

9. The composition of claim 1, wherein said homologs of magnesidin comprise C6 marnesidin and C4 magnesidin.

10. The composition of claim 9, wherein the structure for C6 magnesidin is the following:

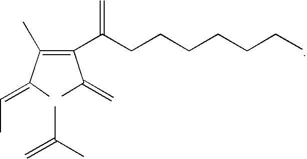

11. The composition of claim 9, wherein the structure for C4 magnesidin is the following:

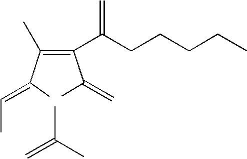

12. A method for producing an anti-microbial agent using a supercritical fluid, comprising the following:
(a) obtaining a sample mass putatively containing an anti-microbial agent;
(b) subjecting said sample mass of (a) to critical conditions for carbon dioxide, wherein said critical conditions include a temperature of about 40° C. and a pressure of about 3000 psig; and
(c) collecting fractions from step (b).

13. The method of claim 12, wherein said sample mass is Vibrio gazogenes.

14. The method of claim 12, wherein said anti-microbial agent is selected from the group consisting of prodigiosin, magnesidin and homologs thereof.

15. The method of claim 12 further comprising the use of an entrainer such as alcohol in step (b).

16. The method of claim 15, wherein said entrainer is methanol.

17. A method of reducing dental plaque formation in an individual by contacting said individual's oral cavity with an anti-microbial agent selected from the group consisting of prodigiosin, magnesidin and homologs thereof.

18. The method of claim 17, wherein said anti-microbial agent is contained within a dentrifice.

19. The method of claim 17 further comprising the co-administration of an anti-bacterial agent.
